(19) United States
(12) Patent Application Publication
(10) Pub. No.: US 2022/0225927 A1
(43) Pub. Date: Jul. 21, 2022

(54) DEVICE FOR SKIN CONDITION MEASUREMENT AND SKIN CARE, AND SYSTEM FOR SKIN CONDITION MEASUREMENT AND SKIN CARE COMPRISING SAME

(71) Applicant: LULULAB INC., Seoul (KR)

(72) Inventors: Sang Wook YOO, Suwon-si, Gyeonggi-do (KR); Yong Joon CHOE, Seoul (KR)

(73) Assignee: LULULAB INC., Seoul (KR)

(21) Appl. No.: 17/611,571

(22) PCT Filed: May 19, 2020
(86) PCT No.: PCT/KR2020/006532

§ 371 Date: Nov. 16, 2021

(30) Foreign Application Priority Data
May 20, 2019 (KR) 10-2019-0058750

(51) Int. Cl. A61B 5/00; A61B 5/01; A61L 2/10

(52) U.S. Cl. CPC A61B 5/441; A61L 2/10; A61B 5/01; A61B 5/0077

(57) ABSTRACT

The present invention relates to a device for skin condition measurement and skin care, and a system for skin condition measurement and skin care comprising same. More particularly, the present invention relates to a device for skin condition measurement and skin care, and a system for skin condition measurement and skin care comprising same which are designed to measure the skin condition and provide a guide for the type of skin care equipment, located in an inner space, and the instructions therefor.

### TECHNICAL FIELD

The present invention relates to a device for skin condition measurement and skin care, and a system for skin condition measurement and skin care including the same.

More specifically, the present invention relates to a device for skin condition measurement and skin care, and a system for skin condition measurement and skin care including the same, which are designed to measure the skin condition and provide a guide for the type of skin care equipment located in an inner space, and the instructions therefor.

### BACKGROUND ART

In these days, interest in skin care of a user's face is increasing as interest in beauty increases. In particular, a portable skin condition measuring device is being developed to analyze various skin troubles on the user's face (for example, wrinkles, pores and acne) by photographing the user's facial skin.

Recently, a skin condition measurement device which, for example, recommends cosmetics based on skin condition measurement results is used as one component of a skin comprehensive care system. Particularly, a device for measuring skin conditions and recommending customized products is provided based on the development of IoT technology and the accumulation of big data. The present invention relates to a device for skin condition measurement and skin care, and a system for skin condition measurement and skin care including the same.

More specifically, the present invention relates to a device for skin condition measurement and skin care, and a system for skin condition measurement and skin care including the same, which are designed to measure the skin condition and provide a guide for the type of skin care equipment located in an inner space, and the instructions therefor. The research and development on the system is being actively conducted.

However, at the current technical stage, there is no case of commercialized technology that measures the skin condition and provides skin care equipment with controlled type or operation mode based on the measurement.

(Patent Document 1) Korean Patent Publication No. 2005-0063781

### DISCLOSURE

### Technical Problem

The present invention provides a device for skin condition measurement and skin care, which is designed to provide a skin condition measurement result by analyzing the user face image obtained by a user face-photographing camera, and recommend a type, a driving set value, and a using method of the care equipment located in an inner space.

In addition, the present invention provides a device for skin condition measurement and skin care to control the care equipment such that the driving set value of the care equipment recommended by the skin condition measurement result is implemented when the care equipment is operated, thereby ensuring the connection between the skin condition measurement and the skin care.

In addition, the present invention provides a device for skin condition measurement and skin care to more accurately measure the user skin condition by analyzing a single user face image or analyzing entire user face images.

In addition, the present invention provides a device for skin condition measurement and skin care, a system for skin condition measurement and skin care including care equipment located in an inner space of the device, and a method for skin condition measurement and skin care using the system.

### Technical Solution

In order to solve the above technical problems, the present invention provides a device for skin condition measurement and skin care, and a system including the same and a method for skin condition measurement and skin care using the same.

The device for skin condition measurement and skin care, which is configured to perform skin condition measurement and recommend at least one of care equipment located in an inner space according to a result of the skin condition measurement, includes: a sensor unit for detecting a user located within a predetermined distance; a light source unit including a plurality of light sources; a user face-photographing camera for photographing a user face image to measure a skin condition of the user; a processor designed to provide a skin condition measurement result by analyzing the user face image obtained by the user face-photographing camera, and recommend a type of the care equipment and a driving set value located in the inner space; a front opening/closing door including a display unit for providing information corresponding to any one of a standby mode, an operation mode, a skin condition measurement mode, and a skin condition care mode; and the inner space having a space disposed with the care equipment, and including a charging terminal designed to charge the care equipment. In addition, the processor may control the care equipment such that the driving set value of the care equipment recommended by the skin condition measurement result is implemented when the care equipment is operated.

In one embodiment, the sensor unit may include: a first sensor for detecting an object in front of the device; and a second sensor for determining whether the object is the user.

In one embodiment, the sensor unit may further include a third sensor for checking the temperature change of the user face in real time.

In one embodiment, the processor may analyze a single user face image obtained by the user face-photographing camera, or may analyze an entire image of the user face obtained by extracting a specific area of the user face from each of a plurality of user face images obtained based on a plurality of photographing parameters set to extract the specific area of the user face, and combining the extracted specific area of the user face.

In one embodiment, the processor may control the off-operation of the display unit according to switching to the manual mode driven when the recommended care equipment is spaced apart from the device for skin condition measurement and skin care by a predetermined distance or more.

In one embodiment, the display unit of the front opening/closing door may include a half-mirror glass having a transmittance of 30% to 70% for light having a wavelength in a range of 380 nm to 780 nm, or a black touch panel having a transmittance less than 5% for light having a wavelength in a range of 380 nm to 780 nm.

In one embodiment, when the display unit of the front opening/closing door includes the half-mirror glass, the device for skin condition measurement and skin care may be driven by a virtual touch of the user.

In one embodiment, the display unit in the skin condition care mode may provide a using state and a using method of the care equipment, and a real-time skin condition of the user.

In one embodiment, the front opening/closing door may further include a touch panel including a power for manually controlling an on-off operation of the device, and a setting change unit for changing settings of the display unit according to the mode.

In one embodiment, the inner space may further include: an LED display unit for visually identifying the recommended care equipment; and a UV light irradiating unit for sterilizing the care equipment.

In one embodiment, the device for skin condition measurement and skin care may further include a communication module for controlling the care equipment located in the inner space and interworking with external devices.

In addition, the present invention relates to a system for skin condition measurement and skin care. The system for skin condition measurement and skin care includes: a device for skin condition measurement and skin care; and at least one care equipment located in an inner space of the device.

In addition, the present invention relates to a method for skin condition measurement and skin care. The method for skin condition measurement and skin care includes: detecting a user located within a predetermined distance; obtaining a user face image for measuring a skin condition of the user by using a user face-photographing camera while irradiating a light source; analyzing the user face image obtained by the user face-photographing camera to provide a skin condition measurement result, and recommending a type and a driving set value of care equipment located in an inner space;

controlling the care equipment such that the driving set value of the care equipment recommended by the skin condition measurement result is implemented when the care equipment is operated; and providing the user with a using state and a using method of the care equipment.

### Advantageous Effects

The device for skin condition measurement and skin care according to the present invention can measure the user skin condition more accurately, recommend care equipment located in the inner space of the device based on the measurement, and specifically instruct a using method, so that the user skin condition can be improved.

In particular, the device for skin condition measurement and skin care according to the present invention recommends a type and a driving set value of care equipment located in an inner space according to the skin condition measurement result, and controls the care equipment such that the driving set value of the care equipment is implemented based on the recommendation when the care equipment is operated, so that the connection between skin condition measurement and care process can be excellent.

Thus, when the system and method including the device for skin condition measurement and skin care according to the present invention are used, the user skin condition can be measured, and a corresponding type of the skin care equipment, a driving set value, and a specific using method of the care equipment can be effectively linked and transferred.

It is apparent that the scope of the present invention is not limited by the above advantageous effects.

### DESCRIPTION OF DRAWINGS

FIG. 1 is one example of a front view of the device for skin condition measurement and skin care according to the present invention.

FIGS. 2 to 6 and 8 show examples of a screen of a display unit according to each mode of the device for skin condition measurement and skin care according to the present invention.

FIG. 7 shows one example of an inner space of the device for skin condition measurement and skin care according to the present invention.

### BEST MODE

### Mode for Invention

Hereinafter, the present invention will be described in more detail with reference to drawings and examples.

In the specification herein, a singular expression includes a plural expression unless otherwise specified.

For the terms used herein, recently widely used general terms are selected as possible in consideration of the function according to the present invention, however, the term may vary depending on the intention of those skilled in the art, judicial cases, the advent of new technology, or the like. In addition, in a certain case, the term may be arbitrarily selected by the applicant, and in the above case, the meaning thereof will be described in detail in the relevant description of the invention. Therefore, the term used in the present invention should be defined based on the meaning of the term and contents throughout the present invention, not simply on the name of the term.

The present invention may be applied with various modifications and have various embodiments, and specific embodiments will be illustrated in the drawings and described in the detailed description in detail. However, it is not intended to limit the scope of the specific embodiment, and it will be understood that the invention is intended to cover all modifications, equivalents and alternatives falling within the scope of spirit and technology of the invention. In the description of the embodiments, a detailed description of relevant known technology incorporated herein will be omitted when it possibly makes the subject matter of the present disclosure unclear.

In this specification, the terms such as first and second may be used to describe various elements, however, the elements will not be limited by the above terms. The terms are used only for the purpose of distinguishing one element from another element.

In the specification herein, the singular expression includes a plural expression unless the context clearly means otherwise.

In the specification herein, it will be understood that the terms such as “include” and “constitute” are intended to designate the presence of feature, number, step, operation, element, component, or a combination thereof recited in the specification, which does not preclude the possibility of the presence or addition of one or more other features, numbers, steps, operations, elements, components, or combinations thereof.

Hereinafter, a device for skin condition measurement and skin care, a system including the same, and a method for skin condition measurement and skin care using the same according to the present invention will be described in more detail with reference to the accompanying drawings.

The present invention relates to a device for skin condition measurement and skin care. The device for skin condition measurement and skin care according to the present invention is designed to measure the user's skin condition, recommend appropriate care equipment therefor, and perform specific guidance on a method of using the care equipment.

The inventors has developed a single device for accurately measuring the user's skin condition, recommending care equipment in the device based on the measurement, and simultaneously guiding a method of using the care equipment, and has completed the present invention.

In particular, the device for skin condition measurement and skin care according to the present invention recommends a type and a driving set value of care equipment located in an inner space according to the skin condition measurement result, and controls the care equipment such that the driving set value of the care equipment is implemented based on the recommendation when the care equipment is operated. Thus, the connection between skin condition measurement and care process is excellent.

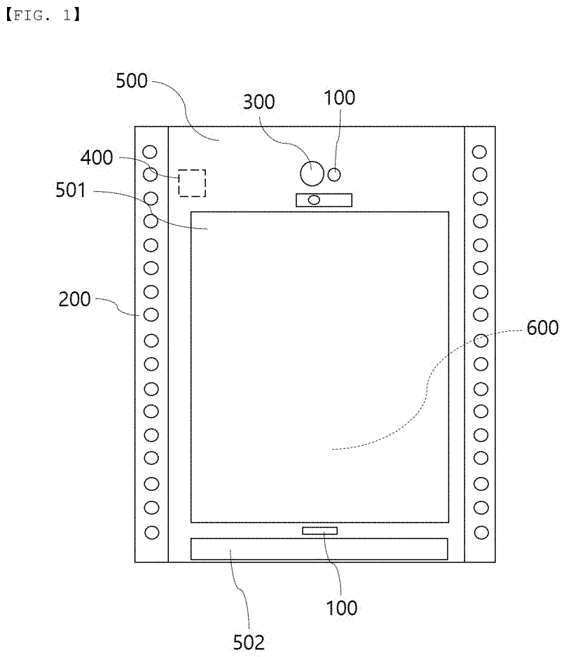

FIG. 1 shows one example of a front view of the device for skin condition measurement and skin care according to the present invention.

As shown in FIG. 1, the device 1 for skin condition measurement and skin care according to the present invention is configure to perform skin condition measurement and recommend at least one of care equipment located in an inner space according to a result of the skin condition measurement, and the device includes: a sensor unit 100 for detecting a user located within a predetermined distance; a light source unit 200 including a plurality of light sources; a user face-photographing camera 300 for photographing a user face image to measure a skin condition of the user; a processor 400 designed to provide a skin condition measurement result by analyzing the user face image obtained by the user face-photographing camera 300, and recommend a type of the care equipment and a driving set value located in the inner space; a front opening/closing door 500 including a display unit 501 for providing information corresponding to any one of a standby mode, an operation mode, a skin condition measurement mode, and a skin condition care mode; and the inner space 600 having a space disposed with the care equipment, and including a charging terminal 601 designed to charge the care equipment. In addition, the processor 400 may control the care equipment such that the driving set value of the care equipment recommended by the skin condition measurement result is implemented when the care equipment is operated.

The device 1 for skin condition measurement and skin care according to the present invention includes a sensor unit 100 for detecting a user located within a predetermined distance.

The sensor unit 100 performs a function of determining whether an object is present in front of the device 1 and whether the object is a user (person).

As shown in FIG. 1, the sensor unit 100 may include a sensor for determining whether an object is present in front of the device 1, and a sensor for determining whether the object is a user (person).

In one embodiment, the sensor unit 100 may include: a first sensor for detecting an object in front of the device 1; and a second sensor for determining whether the object is the user.

The first sensor may be, for example, an optical sensor. The optical sensor serves to detect the presence or absence of an object in front of the device 1, and may be located on any part of a front surface of the device 1.

The second sensor may be, for example, an IR temperature sensor. The IR temperature sensor serves to determine whether the object in front of the device is the user, and may be located on any part of a front surface of the device.

FIG. 1 shown that the first sensor is located at the top or bottom of the front opening/closing door 500, and the second sensor is located at the top or bottom of the front opening/closing door 500. However, this is only an example according to the present invention. Each position of the sensor unit 100 including the first sensor and the second sensor is not limited as shown in FIG. 1.

In addition, the sensor unit 100 may further include a temperature check sensor for checking a temperature change of the user face in real time.

In one embodiment, the sensor unit 100 may further include a third sensor for checking the temperature change of the user face in real time. Through the third sensor, the temperature change of the user face may be checked with an image in real time according to using the care equipment described later, and a person's body temperature may be remotely measured by checking a temperature particularly on a forehead area. The third sensor may be, for example, an IR sensor, but the present invention is not limited thereto.

In addition, the sensor unit 100 may further include a sensor for measuring a body temperature of the user by checking a temperature of a forehead part of the user face.

In one embodiment, the sensor unit 100 may further include a fourth sensor capable of measuring a body temperature by checking a temperature of a forehead part of the user in real time. The fourth sensor may be designed to measure, for example, a temperature of a skin surface above the temporal artery, so as to measure the user's body temperature in real time.

Meanwhile, the sensor unit 100 may further include a distance sensor used to detect a location of the user when obtaining the user face image by the user face-photographing camera 300, or detect the distance between the device 1 and the user for a virtual touch implemented when the front opening/closing door 500 includes the half-mirror glass.

The distance sensor may be, for example, an ultrasonic sensor or a Time of Flight (ToF).

The distance sensor may be used as an element configured to limit a shift of the user's location upon photographing the user face image for measuring the user's skin condition, or implement a virtual touch by sensing a distance between the device 1 and the user.

Accordingly, the distance sensor may enable to prevent the face image from being inaccurately obtained due to deviation of the user face, obtain the user face image that is clearer, and effectively implement the virtual touch.

When it is determined, through the distance sensor, that the user face is separated from the device 1, the device according to the present invention may notify the user face deviation by using the display unit 501 of the front opening/closing door 500 or a separate audio unit in the device 1.

When the distance sensor is a Time of Flight (ToF) sensor, particularly, a 3D ToF sensor, the device 1 according to the present invention may obtain a three-dimensional modeled face image by combining an RGB image and a depth value of the user face image, and additionally implement an air motion detection function that performs a specific action according to a user's motion. In the above case, the processor 400 may be designed to obtain a three-dimensional modeled face image by using the user face images obtained from the 3D ToF sensor, and may be designed to interpret the user's motion and drive a corresponding preset mode.

The device 1 according to the present invention includes a light source unit 200 including a plurality of light sources.

The light source unit 200 includes a plurality of light sources that emit white light or UV light, and may emit light when the user is detected by the sensor unit 100 or emit light upon photographing to obtain the user face image.

The light source unit 200, as shown in FIG. 1 for example, may be located on both sides of the front opening/closing door 500, but the present invention is not limited thereto.

The type and intensity of the light source included in the light source unit 200 may be different according to each mode of the device 1. For example, in the standby mode, the brightness may be implemented to enable the user to perform makeup or the like through the display unit 501 located in the front opening/closing door 500. In the operation mode of obtaining the user face image for skin condition measurement through the user face-photographing camera, the brightness may be improved compared to the standby mode. Meanwhile, in the standby mode, the display unit 501 is required to have an appropriate light transmittance in order for the user to perform makeup or the like through the display unit 501 located in the front opening/closing door 500. Accordingly, in order to implement the above function, the display unit 501 may include the half-mirror glass.

The device 1 according to the present invention includes a user face-photographing camera 300 for photographing a user face image to measure a skin condition of the user.

The user face-photographing camera 300 serves to provide data for skin condition measurement of the user by providing an image obtained by photographing the user face at least once to the processor 400.

The user face-photographing camera 300 may be configured, for example, as a single camera, or may be configured as a dual camera or quad camera. When the user face-photographing camera 300 is configured as the dual or quad camera, a polarizing plate having different polarization axes may be included on a front side, or a polarizing film, UV pass filter or the like may be located on the front side. In this case, the device 1 may implement a function of obtaining a polarized image and a UV image according to a change in photographing mode.

The device 1 according to the present invention includes a processor 400 designed to provide a skin condition measurement result by analyzing the user face image obtained by the user face-photographing camera 300, and recommend a type of the care equipment and a driving set value located in the inner space.

In particular, the device 1 according to the present invention includes a processor 400 designed to recommend customized care equipment based on the analyzed skin condition measurement result, and recommend a driving set value of the care equipment, in addition to simply providing the skin condition measurement result. Thus, the connection between the skin condition measurement and the skin care is excellent.

The processor 400 may control the overall driving and operating of the device in connection with various configurations in the device.

The processor 400 analyzes the user face image obtained by the user face-photographing camera 300.

In one embodiment, the processor 400 may analyze a single user face image obtained by the user face-photographing camera 300, or analyze an entire image of the user face obtained by extracting a specific area of the user face from each of a plurality of user face images obtained based on a plurality of photographing parameters set to extract the specific area of the user face, and combining the extracted specific area of the user face.

The term “single user face image” used herein refers to a single image obtained by a camera for skin condition analysis and analyzed by a processor. The above term is used in the present specification to differentiate the term from the entire user face image formed through a technology of extracting and combining a specific area in a plurality of user face images obtained based on a plurality of photographing parameters.

The term “entire user face image” used herein refers to a user face image that is formed through a technology of extracting and combining a specific face area from each of a plurality of user face images obtained based on a plurality of photographing parameters, and is used for skin condition measurement.

In one embodiment, the processor 400 may analyze a single user face image that is a user face image obtained by photographing a user face based on arbitrary photographing parameters. In the above case, the processor 400 may control the photographing parameters of the user face-photographing camera 300 and the brightness of the light source unit 200.

Specifically, the processor 400 may analyze the brightness of the user face image obtained based on preliminary photographing parameters, and set the preliminary photographing parameters as the photographing parameters. The preliminary photographing parameters may be determined according to an external lighting environment. In the above case, the processor 400 may obtain a user face image based on the photographing parameters set based on the preliminary photographing parameters, and may set the user face image as a single user face image for skin condition measurement.

In addition, the processor 400 may extract a specific area of a user face from each of a plurality of user face images obtained by photographing the user face based on a plurality of photographing parameters, and analyze an entire image of the user face obtained by combining the extracted specific area of the user face.

Specifically, the processor 400 may form an entire user face image for skin condition measurement by extracting a specific area of the user face from each of a plurality of user face images obtained based on a plurality of photographing parameters set to extract the specific area of the user face, and combining the extracted specific area of the user face, and may set the formed image as an entire user face image for skin condition measurement.

The photographing parameters for analyzing the user face skin are set by the processor 400. The photographing parameters, for example, may be the intensity, type and/or direction of the light source included in the light source unit 200, or may be a shutter speed and/or an aperture value of the user face-photographing camera 300.

In one embodiment, the photographing parameters may include at least one of the intensity of the light source, the direction of the light source, the shutter speed of the camera, and the aperture value.

Hereinafter, operations of the processor 400, which is designed to form the entire user face image for skin condition measurement by extracting and combining the specific area of the user face based on a plurality of preliminary photographing parameters and then analyze the formed image, will be described in more detail.

The processor 400 is designed to analyze the brightness of a plurality of user face images obtained based on a plurality of preliminary photographing parameters, and set at least one of the preliminary photographing parameters as the photographing parameters.

In one embodiment, when an image photographed based on a first preliminary photographing parameter among the preliminary photographing parameters has a brightness value in a preset range with respect to a first area (for example, a central area of the face) of the user face, the processor 400 may be designed to set the first preliminary photographing parameter as the first photographing parameter corresponding to the first area of the user face. In addition, when an image photographed based on a second preliminary photographing parameter among the preliminary photographing parameters has a brightness value in a preset range with respect to a second area (for example, a peripheral area of the face) of the user face, the processor may be designed to set the second preliminary photographing parameter as the second photographing parameter corresponding to the second area of the user face.

In other words, the processor 400 may be designed to set a first preliminary photographing parameter, which is configured to have a brightness value in a preset range with respect to the first area of the user face image, as the first photographing parameter, and set a second preliminary photographing parameter, which is configured to have a brightness value in a preset range with respect to the second area of the user face image, as the second photographing parameter.

Based on the photographing parameters, the processor 400 is designed to extract the specific area of the user face from each of the user face images obtained based on the set photographing parameters.

In one embodiment, the processor 400 may be designed to obtain a first user face image by photographing a user face based on the first photographing parameter, and obtain a second user face image by photographing the user face based on the second photographing parameter. In addition, the processor may be designed to extract a user face area corresponding to each of the images from the images, and obtain an entire face image by combining the extracted user face areas.

In other words, the processor 400 may be designed to extract a first face area from the first user face image obtained by the first photographing parameter, and extract a second face area from the second user face image obtained by the second photographing parameter. In addition, the processor may be designed to form the entire user face image for skin condition measurement by combining the first face area and the second face area.

When the first face area and the second face area are combined, the processor 400 may be designed to perform a brightness correction to combine the first face area and the second face area.

Specifically, the processor 400 may be designed to determine a brightness difference value between the first face area and the second face area near a boundary between the first face area and the second face area, and the processor 400 may be designed to determine a combination weight for at least one of the first face area and the second face area based on the brightness difference value. The combination weight may refer to a weight for adjusting the brightness. In addition, the processor may be designed to adjust the brightness of at least one of the first face area and the second face area based on the combination weight, and then combine the areas.

Through the configuration of the processor 400 as described above, the entire user face image for more accurate skin condition measurement can be obtained, and based on this, the skin condition can be effectively analyzed.

The processor 400 is designed to provide the skin condition measurement result to the user after analyzing the user face image, and recommend a type of the care equipment and a driving set value located in the inner space.

The skin condition measurement result of the processor 400, for example, may include an overview indicating the skin condition for each face part, a skin age, a skin score, a skin trouble, and a score for each skin condition diagnosis factor, but the present invention is not limited thereto.

The skin condition measurement of the processor 400, for example, may be implemented based on skin condition measurement data stored in a memory unit of the device. Regarding the skin condition measurement data stored in the memory unit, because latest skin condition measurement information provided by a big data unit of an external server is updated in real time, an accurate skin condition measurement result may be provided to the user.

The processor 400 is designed to recommend a type of the care equipment and a driving set value located in the inner space, together with the skin condition measurement result.

At least one care equipment 2 is located in the inner space 600 of the device according to the present invention, and the processor 400 recommends any one of the care equipment 2 located in the inner space 600, based on the skin condition measurement result. In addition, the processor 400 is designed to recommend the driving set value of the recommended care equipment 2.

In one embodiment, the processor 400 may be designed to provide at least one of the care equipment 2, which is located in the inner space 600 of the device and improving the skin condition based on the skin condition measurement result, on the display unit 501, and recommend a driving set value including the using time, using intensity, using mode, and the like of the provided care equipment 2.

Further, the processor 400 included in the device 1 according to the present invention controls the care equipment 2 so that the recommended care equipment 2 may be driven according to the driving set value.

In other words, the processor 400 controls the care equipment 2 such that the driving set value of the care equipment recommended by the skin condition measurement result is implemented when the care equipment 2 is operated.

The device 1 according to the present invention controls the driving set value during the initial operation of the care equipment 2 to correspond to the skin condition measurement result in addition to simply diagnosing skin conditions and recommending care products, so that the connectivity between skin condition measurement and care process and the skin condition improvement effect can be ensured.

The control of the processor 400 for the care equipment 2, for example, may be performed by a communication module described later. Specifically, the door opening of the front opening/closing door 500 is detected by the door sensor of the device 1 and/or the door opening is detected by a signal strength variation of a Bluetooth Low Energy, and then data on the driving set value is BLE advertised and transmitted to the care device 2 from the device 1, so that the care equipment 2 may be controlled such that the driving set value of the care equipment 2 set in the processor 400 may be implemented when the care equipment 2 is operated.

The processor 400 may control the user interface (UI) provided on the display unit 501 of the front opening/closing door 500 according to the standby mode, the operation mode, the skin condition measurement mode and the skin condition care mode, the intensity and the direction of the light source included in the light source unit 200, and the shutter speed and the aperture value of the user face-photographing camera 300.

In addition, the processor 400 may recognize a state, in which the user uses the recommended care equipment 2 at a predetermined separation distance or more, as a manual mode, and control an off-operation of the display unit included in the front opening/closing door 500.

In one embodiment, the processor 400 may control the off-operation of the display unit 500 according to switching to the manual mode driven when the recommended care equipment is spaced apart from the device for skin condition measurement and skin care by a predetermined distance or more.

In addition, the processor 400 may control an on-off operation of an LED display unit 602 that enables the user to visually identify the recommended care equipment 2.

In one embodiment, after detecting the door opening of the front opening/closing door 500, the processor 400 may control the on-off operation of the LED display unit 602 that enables the user to visually identify the recommended care equipment 2. Accordingly, the user may easily identify the recommended care equipment 2.

In addition, the processor 400 may be designed to specifically provide the user with a using state and a using method of the recommended care equipment 2 through the display unit 501.

More specifically, the processor 400 may be designed to provide the user with a real-time usage state of the user through the display unit 501 when the user uses the recommended care equipment 2, and may be designed to provide the user, through the display unit 501, with a specific using method including a using portion and a location of the recommended care equipment 2. In the above case, the processor 400 may implement the above configuration by controlling the operations of the light source unit 200, the user face-photographing camera 300, and the display unit 501.

In addition, the processor 400 may be designed to provide a real-time skin condition of the user using the recommended care equipment 2 through the display unit 501.

More specifically, the processor 400 may be designed to provide, through the display unit 501, changes in skin condition of the user, for example, changes in skin condition including changes in temperature for each part of the face upon or after the user's use of the recommended care equipment 2. In the above case, the processor 400 may control the touch panel, the sensor unit 100, the light source unit 200, the user face-photographing camera 300, and the display unit 501 to implement the above configuration when the user manipulates the setting change unit of the touch panel.

The device 1 according to the present invention includes a front opening/closing door 500 including a display unit 501.

The front opening/closing door 500 serves to provide information for each mode to the user via the display unit 501, and has an opened and closed structure for allowing the user to take out and use the recommended care equipment 2 located in the inner space 600.

The front opening/closing door 500 includes the display unit 501, in which the display unit 501 provides the user with information corresponding to any one of the standby mode, the operation mode, the skin condition measurement mode, and the skin condition care mode.

The term “standby mode” used herein refers to a mode state of the device 1 before the sensor unit 100 of the device 1 recognizes the user, and signifies to a mode of providing daily life information including date, day of the week, weather and fine dust index.

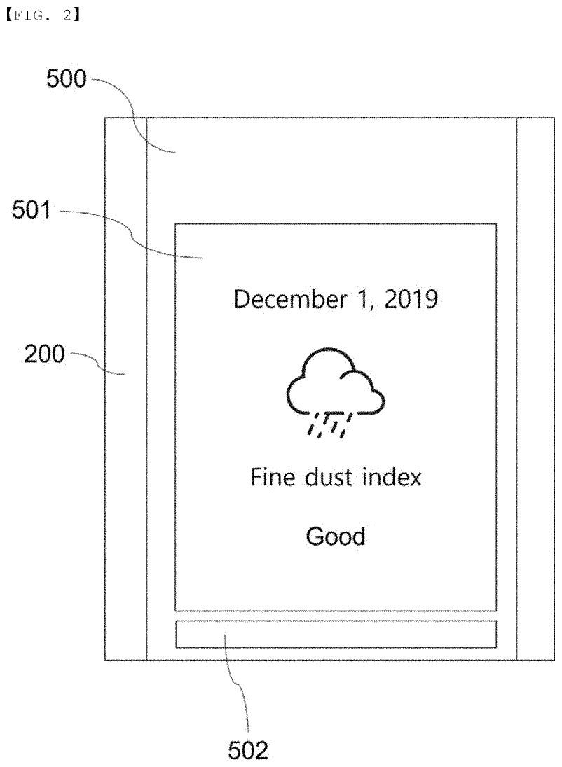

The standby mode, as shown in FIG. 2 for example, may signify a mode state of providing the living information including date, weather, and fine dust index before the device 1 recognizes the user.

The term “operation mode” used herein signifies to a mode in which the display unit 501 of the front opening/closing door 500 provides the UI and content by a touch (including virtual touch) action of the user when the sensor unit 100 of the device 1 recognizes the user.

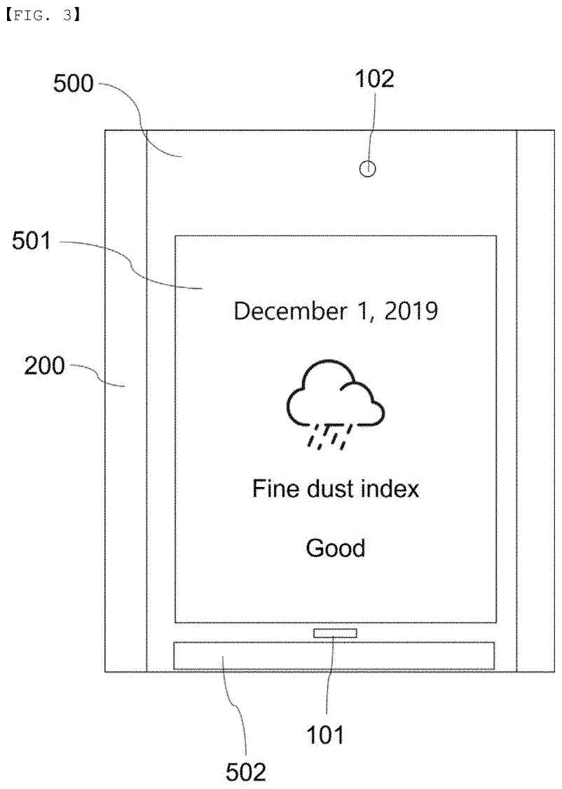

The operation mode, as shown in FIG. 3 for example, may signify a mode activated when the object in front of the device is detected by a first sensor 101 of the sensor unit 100, the second sensor 102 determines whether the object is a user, and the object is determined as a user.

The term “skin condition measurement mode” used herein signifies to a mode of driving the light source unit 200 and the user face-photographing camera 300 to diagnose the user skin condition, so that the user skin condition is diagnosed.

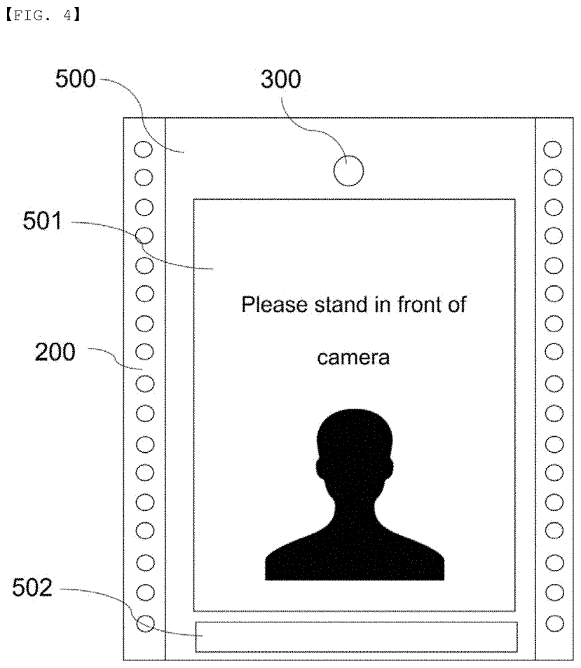

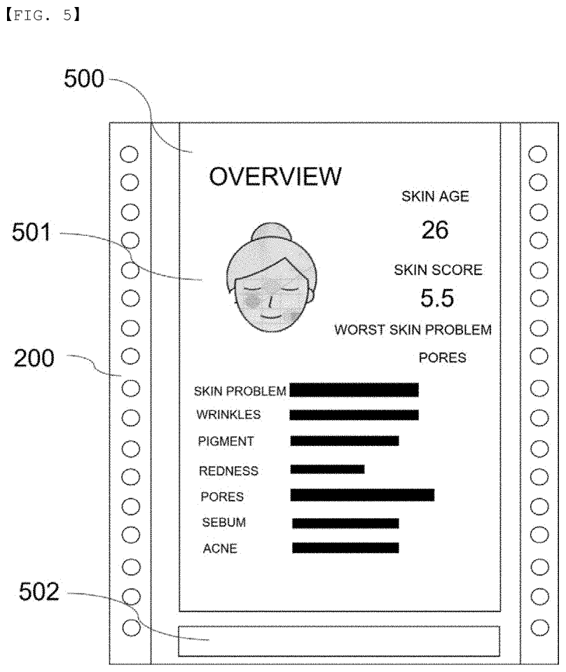

The skin condition measurement mode, as shown in FIGS. 4 and 5 for example, may signify a mode that photographs the user face via the user face-photographing camera 300 together with instructions on photographing content of the display unit 501 (FIG. 4), analyzes the user face image via the processor 400, and provides the skin condition measurement result including a skin condition for each face part, a skin age, a skin score, a skin trouble, and a score for each skin condition diagnosis factor.

The term “skin condition care mode” used herein signifies to a mode that recommends and guides suitable care equipment 2 based on the measured skin condition of the user, presents the specific using state and using method therefor, and provides a real-time change status according to usage.

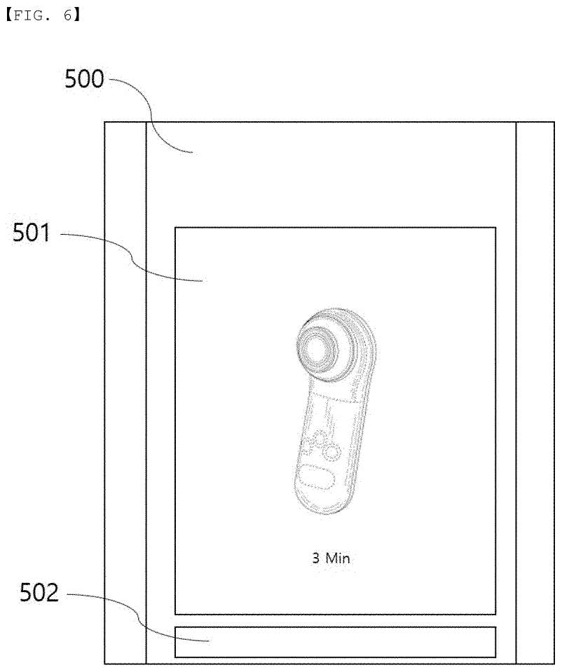

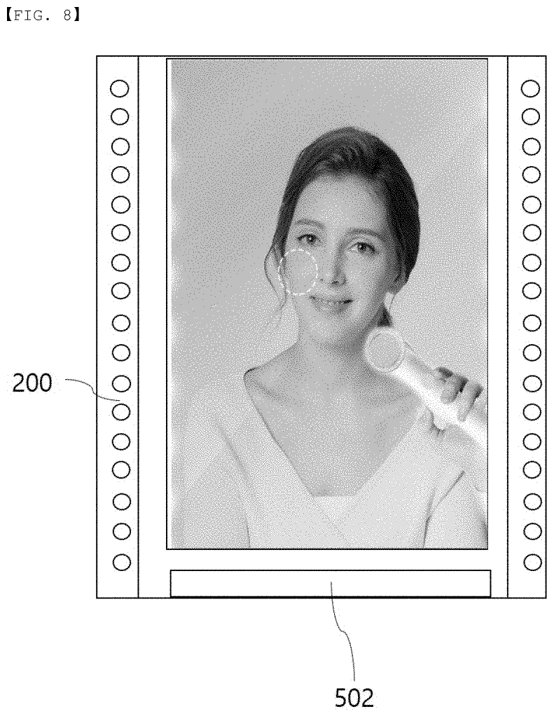

The skin condition care mode may signify a mode that recommends any one of the care equipment 2 located in the inner space 600 on the UI of the display unit 501 as shown in FIG. 6 for example, and performs specific instructions on the using state and using method as shown in FIG. 8.

The display unit 501 included in the front opening/closing door 500 is driven by a user's touch, and the touch may include a physical touch and a virtual touch.

The display unit 501 may be a translucent display that enables the user to visually identify a shape of the care device 2 therein, or may be an opaque touch display.

In one embodiment, the display unit 501 of the front opening/closing door 500 may include a half-mirror glass having a transmittance of 30% to 70% for light having a wavelength in a range of 380 nm to 780 nm, or a black touch panel having a transmittance less than 5% for light having a wavelength in a range of 380 nm to 780 nm.

When the display unit 501 includes the half-mirror glass, the user, through virtual touch actions, may control the driving and detailed UI of the operation mode, skin condition measurement mode, and skin condition care mode of the device 1, and content operation. In other words, when the display unit 501 of the front opening/closing door 500 includes the half-mirror glass, the device 1 is driven by the user's virtual touch.

Meanwhile, when the display unit 501 includes the black touch panel, the user, through physical touch actions on the display unit 501, may control the driving and detailed UI of the operation mode, skin condition measurement mode, and skin condition care mode of the device 1, and content operation.

The display unit 501 may provide the user with different UIs according to the above-described modes.

In one embodiment, the display unit 501 in the skin condition care mode may provide a using state and a using method of the care equipment, and a real-time skin condition of the user. The provision of the using method may include, for example, specifically instructing the using part and the using method of the care equipment 2 while providing display unit 501 with a state in which the user is actually using the care equipment 2. The provision of the real-time user skin condition may include, for example, providing the display unit 501 with changes in temperature for each part in the user face.

The different UIs of the display unit 501 for the using state, the using method, and the real-time skin condition may be implemented by the user action of touching the display unit 501 (including virtual touch), or implemented through a setting change unit included in a separate bottom touch panel.

In addition, the front opening/closing door 500 may further include a touch panel 502 including a power for manually controlling an on-off operation of the device 1, and a setting change unit for changing settings of the display unit 501 according to the mode.

As shown in FIG. 1, the touch panel 502 may be located at the bottom of the display unit 501 to function to manually control the on-off operation of the device 1 itself, and may serve to change the settings of the display unit 501 according to the UI or mode of the display unit 501 via the processor 400 by the user's touch action. The touch panel 502 may be implemented by the user's physical touch action, or implemented by the virtual touch action.

The touch panel 502 may further include a speaker that provides voices with respect to the instruction on a location shift of the user or the text information provided on the UI of the display unit 501.

The device 1 according to the present invention includes the inner space 600 having a space disposed with the care equipment 2, and including a charging terminal 601 designed to charge the care equipment 2.

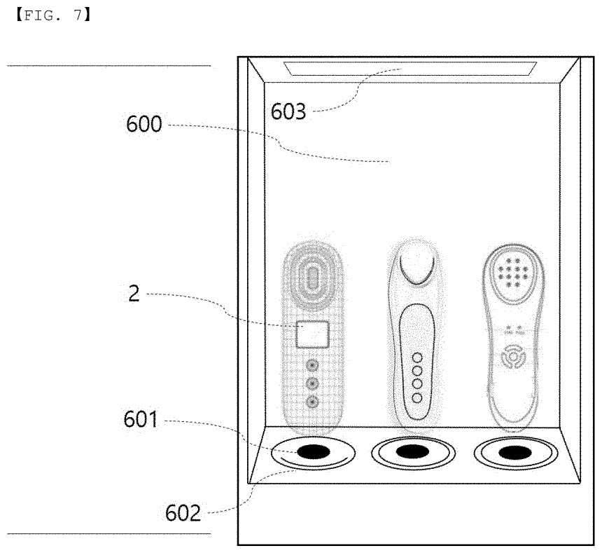

As shown in FIG. 7, the inner space 600 may have a space disposed therein at least one care equipment 2, and a charging terminal 601 corresponding to each care equipment 2 may be located at the bottom of each care equipment 2.

In addition, the inner space 600 may further include a configuration for enabling the user to easily find the care equipment 2 or improving the cleanliness of the care equipment 2.

In one embodiment, the inner space 600 may further include: an LED display unit 602 for visually identifying the recommended care equipment 2; and a UV light irradiating unit 603 for sterilizing the care equipment 2.

Although FIG. 7 shows that the LED display unit 602 is located near the charging terminal 601, this is only an example according to the present invention. The position of the LED display unit 602 is not particularly limited as long as the condition for the function of the LED display unit 602, specifically, the function of enabling the user to find the care equipment 2 more easily is achieved.

In addition, although FIG. 7 shows that the UV light irradiating unit 603 for sterilizing the care equipment 2 is located at the top of the inner space 600, this is only an example according to the present invention. The position of the UV light irradiating unit 603 is not particularly limited as long as the condition for the function of the UV light irradiating unit 603, specifically, the function of sterilizing the care equipment 2 is achieved.

The user may perform the skin care after selecting the care equipment 2 recommended through the display unit 501 and visually identified through the LED display unit 602 among the care equipment 2 located in the inner space 600, and check the using state and the using method, and the real-time skin condition change according to the instruction of the display unit 501.

Meanwhile, the care equipment 2 located in the inner space 600 is controlled by the driving set value allocated by the processor 400 of the device 1 and provided to the display unit 501 while interworking with the device 1. The device 1 and the care equipment 2 may be connected through a communication module to implement the above-described control.

In one embodiment, the device 1 according to the present invention may further include a communication module for controlling the care equipment 2 located in the inner space 600 and interworking with external devices.

The communication module may serve to wirelessly connect the device 1 to the care equipment 2 so as to enable the device 1 to control the care equipment 2, and may serve to transmit various information on the user skin condition measurement obtained from the device 1 or control operations of the device 1 by the user's remote control while interworking with the device 1 and the external device, for example, an application in a PC, external server, or portable terminal.

In one embodiment, the communication module may include, as an example, a wireless communication module, such as Wi-Fi, ZigBee communication module, Bluetooth and Bluetooth Low Energy, and/or a wired communication module, but the present invention is not limited thereto.

In a more specific embodiment, the device 1 and the care equipment 2 may interwork with each other via the wireless communication module such as Bluetooth and Bluetooth Low Energy, and the device 1 and the external device may interwork with each other through Wi-Fi or the wired communication module.

In addition, the present invention relates to a system for skin condition measurement and skin care, which includes: a device 1 for skin condition measurement and skin care; and care equipment 2 located in an inner space 600 of the device 1.

The system for skin condition measurement and skin care includes: a device 1 for skin condition diagnosis and skin care; and at least one care equipment 2 located in an inner space 600 of the device 1.

The system including the device for skin condition measurement and skin care according to the present invention can measure the user skin condition, and effectively link and transfer a corresponding type of the skin care equipment, a driving set value, a specific using method of the care equipment, and a real-time using state.

In addition, the present invention relates to a method for skin condition measurement and skin care using the system for skin condition measurement and skin care.

The method includes: detecting a user located within a predetermined distance; obtaining a user face image for diagnosing a skin condition of the user by using a user face-photographing camera while irradiating a light source; analyzing the user face image obtained by the user face-photographing camera to provide a skin condition diagnosis result, and recommending a type and a driving set value of care equipment located in an inner space; controlling the care equipment such that the driving set value of the care equipment recommended by the skin condition diagnosis result is implemented when the care equipment is operated; and providing the user with a using state and a using method of the care equipment.

According to the method for skin condition measurement and skin care according to the present invention, the user skin condition can be measured more accurately, the type and driving set value of the care equipment 2 can be recommended based on the above measurement, and the specific using state, using method, and real-time using effect of the care equipment 2 can be visually provided to the user, so that the skin condition measurement and skin care can be unified in one place.

The step of detecting the user located within the predetermined distance is performed by the sensor unit 100 of the device 1.

Specifically, when the user is located in front of the device 1 within the predetermined distance, the first sensor 101 may primarily detect an object and the second sensor 102 may detect the user. When the object is finally determined as the user, the device 1 may release the standby mode and be switched to the operation mode.

The step of obtaining a user face image for diagnosing a skin condition of the user by using a user face-photographing camera while irradiating a light source may be performed by the light source unit 200 including a light source in the device 1, the user face-photographing camera 300, and the processor 400 for controlling the light source unit 200 and the camera 300. In the above step, the device 1 may be switched to the skin condition measurement mode to obtain the user face image for measuring the user skin condition.

The step of analyzing the user face image obtained by the user face-photographing camera to provide a skin condition diagnosis result, and recommending a type and a driving set value of care equipment located in an inner space may be performed by the processor 400 and the display unit 501 controlled by the processor 400. The above step may be implemented in the skin condition measurement mode and the skin condition care mode.

The step of controlling the care equipment such that the driving set value of the care equipment recommended by the skin condition diagnosis result is implemented when the care equipment is operated may be implemented to include, for example, BLE-advertising and transmitting the driving set value data to the care device 2 from the device 1 after detecting the door open of the front opening/closing door 500.

The step of providing the user with a using state and a using method of the care equipment may be performed before and/or simultaneously when the user uses the care equipment 2 located in the inner space 600 of the device 1.

In addition, the method for skin condition measurement and skin care according to the present invention may further include steps of providing the real-time user skin condition after the step of providing the user with a using state and a using method of the care equipment.

As described above, although the present disclose has been described by the limited embodiments and drawings, the present disclosure is not limited to the above embodiments, and it will be understood by those skilled in the art that various changes and modifications are available from the above-mentioned description. Therefore, the scope of the present disclosure will not be limited to the aforementioned embodiments, and will be determined by the following claims and the equivalents thereof.

### DESCRIPTION OF REFERENCE NUMERALS

| | |
|---|---|
| 1: Device for skin condition measure- | |
| ment and skin care | |
| 2: Care equipment | 100: Sensor unit |
| 200: Light source unit | |
| 300: User face-photographing camera | |
| 400: Processor | 500: Front opening/closing door |
| 501: Display unit | 502: Touch panel |
| 600: Inner space | 601: Charging terminal |
| 602: LED display unit | 603: UV light irradiating unit |

1. A device for skin condition measurement and skin care to perform skin condition measurement and recommend at least one of care equipment located in an inner space according to a result of the skin condition measurement, the device comprising:
a sensor unit for detecting a user located within a predetermined distance;
a light source unit including a plurality of light sources;
a user face-photographing camera for photographing a user face image to measure a skin condition of the user;
a processor designed to provide a skin condition measurement result by analyzing the user face image obtained by the user face-photographing camera, and recommend a type of the care equipment and a driving set value located in the inner space;
a front opening/closing door including a display unit for providing information corresponding to any one of a standby mode, an operation mode, a skin condition measurement mode, and a skin condition care mode; and
the inner space having a space disposed therein with the care equipment, and including a charging terminal designed to charge the care equipment, wherein
the processor controls the care equipment such that the driving set value of the care equipment recommended by the skin condition measurement result is implemented when the care equipment is operated.

2. The device of claim 1, wherein the sensor unit includes:
a first sensor for detecting an object in front of the device; and
a second sensor for determining whether the object is the user.

3. The device of claim 2, wherein the sensor unit uses the second sensor or further includes an additional third sensor to check a temperature change of the user face in real time.

4. The device of claim 1, wherein the processor analyzes a single user face image obtained by the user face-photographing camera, or analyzes an entire image of the user face obtained by extracting a specific area of the user face from each of a plurality of user face images obtained based on a plurality of photographing parameters set to extract the specific area of the user face and combining the extracted specific area of the user face.

5. The device of claim 1, wherein the processor controls an off-operation of the display unit according to switching to a manual mode driven when the recommended care equipment is spaced apart from the device for skin condition measurement and skin care by a predetermined distance or more.

6. The device of claim 1, wherein the display unit of the front opening/closing door includes a half-mirror glass having a transmittance of 30% to 70% for light having a wavelength in a range of 380 nm to 780 nm, or a black touch panel having a transmittance less than 5% for light having a wavelength in a range of 380 nm to 780 nm.

7. The device of claim 6, wherein the display unit of the front opening/closing door is driven by a virtual touch of the user when including the half-mirror glass.

8. The device of claim 1, wherein the display unit in the skin condition care mode provides a using state and a using method of the care equipment, and a real-time skin condition of the user.

9. The device of claim 1, wherein the front opening/closing door further includes a touch panel including a power for manually controlling an on-off operation of the device, and a setting change unit for changing settings of the display unit according to the mode.

10. The device of claim 1, wherein the inner space further includes:
an LED display unit for visually identifying the recommended care equipment; and
an UV light irradiating unit for sterilizing the care equipment.

11. The device of claim 1, further comprising:
a communication module for controlling the care equipment located in the inner space, and interworking with external devices.

12. A system for skin condition measurement and skin care, the system comprising:
a device for skin condition measurement and skin care according to claim 1; and
at least one care equipment located in an inner space of the device.

13. A method for skin condition measurement and skin care using a system according to claim 11, the method comprising:
detecting a user located within a predetermined distance;
obtaining a user face image for measuring a skin condition of the user by using a user face-photographing camera while irradiating a light source;
analyzing the user face image obtained by the user face-photographing camera to provide a skin condition measurement result, and recommending a type and a driving set value of care equipment located in an inner space;
controlling the care equipment such that the driving set value of the care equipment recommended by the skin condition measurement result is implemented when the care equipment is operated; and
providing the user with a using state and a using method of the care equipment.
